(19) United States
(12) Patent Application Publication
(10) Pub. No.: US 2019/0321473 A1
(43) Pub. Date: Oct. 24, 2019

(54) MINIMALLY-INVASIVE, HIGH RESOLUTION NEUROMODULATION OF DEEP BRAIN AND CORTICAL STRUCTURES USING CIRCUIT-SPECIFIC PROMOTERS AND FOCUSED ULTRASOUND ARRAY

(71) Applicant: Circuit Therapeutics, Inc., Mountain View, CA (US)

(72) Inventors: Christopher L. Towne, San Francisco, CA (US); Dan E. Andersen, Menlo Park, CA (US)

(73) Assignee: Circuit Therapeutics, Inc., Mountain View, CA (US)

(21) Appl. No.: 16/373,622

(22) Filed: Apr. 2, 2019

Related U.S. Application Data
(60) Provisional application No. 62/651,637, filed on Apr. 2, 2018.

(51) Int. Cl. A61K 41/00; A61N 7/00; A61B 5/055; A61K 38/17

(52) U.S. Cl. CPC A61K 41/0047; A61N 7/00; A61N 2007/0026; A61K 38/1709; A61B 5/055

(57) ABSTRACT

One embodiment is directed to a method for treating the nervous system of a patient, comprising: determining a desired nervous system functional modulation to be facilitated by sonogenetic intervention; selecting targeted neuroanatomy for achieving the desired functional outcome with sonogenetic intervention; and delivering an effective amount of polynucleotide comprising a sound-responsive opsin protein which is expressed in neurons of the targeted neuroanatomy, wherein delivering comprises systemically injecting the effective amount of polynucleotide and facilitating diffusive access to the targeted neuroanatomy using focused ultrasound energy.

### RELATED APPLICATION DATA

The present application claims priority to U.S. Provisional Application Ser. No. 62/651,637, filed Apr. 2, 2018. The foregoing application is hereby incorporated by reference into the present application in its entirety.

### FIELD OF THE INVENTION:

The present invention relates generally to systems and methods for neuromodulation of the central nervous system for treatment of pathological conditions and enhancement of non-pathological neural function.

### BACKGROUND

Recent advances in transcranial focused ultrasound (FUS) technology have permitted non-invasive and targeted blood brain barrier (BBB) opening in several animal models including mice, rats and non-human primates. The technique involves a systemic injection of a mixture composed of ultrasound contrast agents (lipid-based microbubbles) and molecules to be delivered. The emitted ultrasonic waves propagate through the skull and cause the microbubbles to cavitate (to oscillate) within the capillaries in the targeted brain region. This cavitation causes transient loosening of the tight junctions between endothelial cells, which allows BBB opening and the molecules of interest to diffuse into the brain according to their concentration gradient. An example of this has been the delivery of adeno-associated virus (AAV) expressing channelrhodopsin2 (ChR2) to the brain of mice by Wang and colleagues in Scientific Reports (2017) Jan. 6; 7:39955, which is incorporated by reference herein in its entirety.

Sonogenetics is an emerging field that uses ultrasound-sensitive proteins to modulate neural excitability. These are typically mechanosensitive ion channels that are inserted into the cell, commonly using viral vectors or any other method utilized by gene transfer, and are expressed on the cell's membrane. When ultrasound waves are applied, potentially through the skull, the ion transport afforded by the protein changes the function of the cell in an ultrasound-sensitive manner. U.S. Provisional Patent Application No. 62/617,921, entitled “System and Method for SonoGenetic Therapy”, is incorporated by reference herein in its entirety.

### SUMMARY

One embodiment is directed to a method for treating the nervous system of a patient, comprising: determining a desired nervous system functional modulation to be facilitated by sonogenetic intervention; selecting targeted neuroanatomy for achieving the desired functional outcome with sonogenetic intervention; and delivering an effective amount of polynucleotide comprising a sound-responsive opsin protein which is expressed in neurons of the targeted neuroanatomy, wherein delivering comprises systemically injecting the effective amount of polynucleotide and facilitating diffusive access to the targeted neuroanatomy using focused ultrasound energy. The method further may comprise waiting for a period of time to ensure that sufficient portions of the targeted neuroanatomy will express the sound-responsive opsin protein upon exposure to treatment sound energy. The method further may comprise delivering treatment sound energy to the targeted neuroanatomy to cause controlled, specific excitation and/or inhibition by virtue of the presence of sound-responsive opsin protein in such neuroanatomy. Determining a desired nervous system functional modulation to be facilitated by sonogenetic intervention may comprise determining a desired nervous system functional modulation to be facilitated by sonogenetic excitation. Determining a desired nervous system functional modulation to be facilitated by sonogenetic intervention may comprise determining a desired nervous system functional modulation to be facilitated by sonogenetic inhibition. Determining a desired nervous system functional modulation to be facilitated by sonogenetic intervention may comprise determining a desired nervous system functional modulation to be facilitated by sonogenetic excitation and inhibition. Selecting targeted neuroanatomy for achieving the desired functional outcome with sonogenetic intervention may comprise selecting targeted neuroanatomy for achieving the desired functional outcome with sonogenetic excitation. Selecting targeted neuroanatomy for achieving the desired functional outcome with sonogenetic intervention may comprise selecting targeted neuroanatomy for achieving the desired functional outcome with sonogenetic inhibition. Selecting targeted neuroanatomy for achieving the desired functional outcome with sonogenetic intervention may comprise selecting targeted neuroanatomy for achieving the desired functional outcome with sonogenetic excitation and inhibition. Systemically injecting the effective amount of polynucleotide may comprise injecting the effective amount of polynucleotide into a major blood vessel of the patient. Facilitating diffusive access to the targeted neuroanatomy using focused ultrasound energy may comprise utilizing magnetic resonance data to assist in guiding an emitter of focused ultrasound energy. A plan array of focused ultrasound energy delivery points may be created based at least in part upon one or more preoperative images. Focused ultrasound energy may be applied to the patient in accordance with the plan array after completion of the plan array. The plan array may comprise a plurality of access points that are non-coplanar. The plan array may comprise a plurality of access points that represent an access surface. The access surface may be replicated in a direction orthogonal to the access surface to create a multi-layered access volume. Delivering an effective amount of polynucleotide may comprise delivering a protein. Delivering an effective amount of polynucleotide further may comprise packaging the protein with a viral vector. The virus may be selected from the group consisting of: AAV1, AAV2, AAV4, AAV5, AAV6, AAV7, AAV8, AAV9, lentivirus, and HSV. Delivering an effective amount of polynucleotide further may comprise packaging the protein with a promoter. The transcription promoter may be selected from the group consisting of: CaMKIIa, hSyn, CMV, Hb9Hb, Thy1, and Ef1a.

### BRIEF DESCRIPTION OF THE DRAWINGS

FIG. 1A illustrates a conceptual view of a configuration in accordance with the present invention for minimally-invasive, high-resolution neuromodulation of targeted tissue structures.

FIG. 1B illustrates a process configuration in accordance with the present invention for minimally-invasive, high-resolution neuromodulation of targeted tissue structures.

FIGS. 2A and 2B illustrate aspects of one embodiment of retrograde circuit labeling for analysis by single-cell genomics.

FIG. 2C illustrates aspects of an embodiment for utilizing single-cell RNA sequencing data to build a cell atlas for the analysis of circuit-specific gene activity in the brain.

FIGS. 3A-3C illustrate aspects of one embodiment of a work flow for development of therapeutic tools, based on gene expression specificity in neural circuits of the brain.

FIG. 4A illustrates a conceptual view of a configuration for achieving minimally-invasive, high-resolution neuromodulation of a targeted tissue structure, such as the cortex of the brain, using focused ultrasound arrays.

FIGS. 4B-4D illustrate aspects of embodiments for utilizing focused ultrasound to assist in the delivery of one or more sonogenetic proteins.

FIG. 5 illustrates an embodiment of a 96 channel electrode array.

FIG. 6 illustrates aspects of a process flow configuration in accordance with the present invention for minimally-invasive, high-resolution neuromodulation of targeted tissue structures.

### DETAILED DESCRIPTION

By combining FUS-mediated AAV delivery and sonogenetics, there lies the possibility to achieve minimally-invasive neuromodulation. Specifically, an AAV expressing a sonogenetic protein may be systematically introduced, such as by intravenous injection into a major vessel of the patient, such as the femoral vein. The patient then may undergo magnetic-resonance imaging (MRI)-guided FUS on the desired brain region, transiently opening the BBB and delivering the vector. After transduction, the vector expresses the sonogenetic protein in the neurons of the desired brain region. An external, non-implanted, ultrasound transducer can then be used to transmit ultrasound waves through the skull into the brain to activate the sonogenetic protein, modulating neuron behavior.

The combination of FUS-mediated AAV delivery and sonogenetics facilitates minimally-invasive neuromodulation, however, may not provide for sufficiently high resolution neuromodulation. FUS delivery allows expression of AAV to a region within the brain, but does not restrict it to distinct circuits or cell types which would be desirable for an effective treatment of pathological conditions and a targeted enhancement of non-pathological neural function. To achieve heightened clinical resolution, minimally invasive neuromodulation of deep brain structures as well as FUS-mediated sonogenetics may be combined with circuit-specific gene promoters identified through novel bioinformatics techniques. Indeed, single cell genomics including single-cell transcriptomics and epigenomics, the analysis of gene expression and chromatin state respectively, in dozens to millions of single cells, has revolutionized our understanding of the cellular complexity and heterogeneity of the neural system. Usually coupled to next-generation sequencing (NGS) technology, such as for single-cell RNA sequencing (scRNA-Seq), the method allows for efficient analysis of any gene's expression in a cell, and classification of cell types based on their unique gene expression patterns (the transcriptome). Importantly, the method allows for measuring the specificity of a gene for any cell type or—as particularly relevant to this invention—neural circuit of interest and therefore, for discovery of gene regulatory elements (including gene promoters, enhancers, repressors) that can be used for targeting any DNA-encoded agent to a specific cell type or neural circuit for monitoring and/or control of neural network activity.

Viral expression systems have the dual advantages of fast and versatile implementation combined with high copy number for robust expression levels in targeted neuroanatomy. Cellular specificity may be obtained with viruses by virtue of promoter selection if the promoters are small and specific, by localized targeting, and by restriction of sound-responsive opsin protein activation (i.e., via targeted irradiation) of particular cells or projections of cells. In an embodiment, a sound-responsive opsin protein is targeted by methods described in Yizhar et al. 2011, Neuron 71:9-34. In addition, different serotypes of the virus (conferred by the viral capsid or coat proteins) will show different tissue tropism. Lenti- and adeno-associated (“AAV”) viral vectors have been utilized successfully to introduce opsins into the mouse, rat and primate brain. Other vectors include but are not limited to equine infectious anemia virus pseudotyped with a retrograde transport protein (e.g., Rabies G protein), and herpes simplex virus (“HSV”).

Additionally, these have been well tolerated and highly expressed over relatively long periods of time with no reported adverse effects, providing the opportunity for long-term treatment paradigms. Lentivirus, for example, is easily produced using standard tissue culture and ultracentrifuge techniques, while AAV may be reliably produced either by individual laboratories or through core viral facilities. AAV is a preferred vector due to its safety profile, and AAV serotypes 1 and 6 have been shown to infect motor neurons following intramuscular injection in primates. Additionally, AAV serotype 2 has been shown to be expressed and well tolerated in human patients.

Viral expression techniques, generally comprising delivery of DNA encoding a desired sound-responsive opsin protein and promoter/catalyst sequence packaged within a recombinant viral vector have been utilized with success in mammals to effectively transfect targeted neuroanatomy and deliver genetic material to the nuclei of targeted neurons, thereby inducing such neurons to produce sound-responsive proteins which are migrated throughout the neuron cell membranes where they are made functionally available to sonic irradiation components of the interventional system. Typically a viral vector will package what may be referred to as an “expression cassette”, which will contain the sound-responsive opsin protein and a promoter that will be selected to drive expression of the particular opsin within a targeted set of cells. In the case of adeno-associated virus (AAV), the gene of interest (sound-responsive opsin protein) can be in a single stranded configuration with only one sound-responsive opsin protein expression cassette or in a self-complementary structure with two copies of sound-responsive opsin protein expression cassette complementary in sequence with one another and connected by hairpin loops. The self-complementary AAVs are thought to be more stable and show higher expression levels and show faster expression. A various number of serotypes can be used to express the gene of interest, with serotypes varying in their capsid proteins and tissue tropism. Potential AAV serotypes include, but are not limited to, AAV1, AAV2, AAV4, AAV5, AAV6, AAV7, AAV8, and AAV9. The promoter within the cassette may confer specificity to a targeted tissue, such as in the case of the human synapsin promoter (“hSyn”) or the human Thy1 promoter (“hThy1”), which allow protein expression of the gene under its control in neurons. Alternatively, a ubiquitous promoter may be utilized, such as the human cytomegalovirus (“CMV”) promoter, or the chicken beta-actin (“CBA”) promoter, each of which is not neural specific, and each of which has been utilized safely in gene therapy trials for neurodegenerative disease. Another example is the human elongation factor-1 alpha promoter (EF1a), which also allows ubiquitous expression of the gene. Another example are the calmodulin-dependent protein kinase II promoters (e.g. CaMKii, CaMK2A, CaMK2B, CaMK2D, and/or CaMK2G), which allow for targeting of excitatory glutamatergic neurons. Constructs may be optimized for specific cell populations and are not limited to such illustrative examples.

To facilitate high-resolution, minimally invasive neuromodulation of targeted tissue structures, such as cortical structures, FUS may be used to target the AAV to discrete 2 and 3-Dimensional arrays on the cortex or other superficial structures. In previous brain-machine interfaces, multi-channel electrode arrays have been used to activate neurons in the cortex directly, typically with channels from 16 to 96 channels. In one embodiment, rather than direct connectivity with an array of electrodes, FUS may be used to functionally replicate these focal sites, but by precision-delivering AAV rather than electrical current. As the AAV expresses sonogenetic proteins in specific neural circuits at these focal sites, this facilitates multichannel modulation in high resolution to the cortex using transcranial ultrasound. In one variation, therefore, AAV may be injected intravenously, and then FUS may be used to disrupt the BBB at small designated sites in the cortex (such as in a predetermined or prescribed two or three dimensional pattern). After AAV delivery and expression of the sonogenetic protein, transcranial ultrasound then may be targeted at the different foci to induce discrete sonogenetic activation.

The ability to remove the genome of the viral vector after gene delivery provides an embodiment wherein the safety profile of the approach may be enhanced. Further, it may assist with adoption in healthy subjects, such as those in the armed forces, who would seek to use the neuromodulation transiently only for active duty. Thus one embodiment comprises utilization of a drug-inducible promoter, configured to result in excision of the coding region of viral vector genome upon drug delivery. Such a configuration provides for controlled irreversible elimination of expression of the expressed transgene at a desired time.

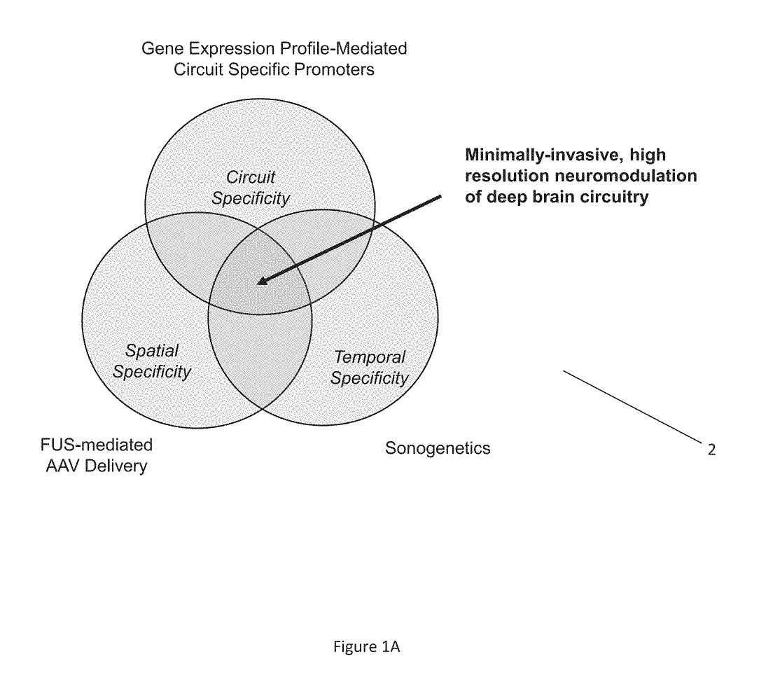

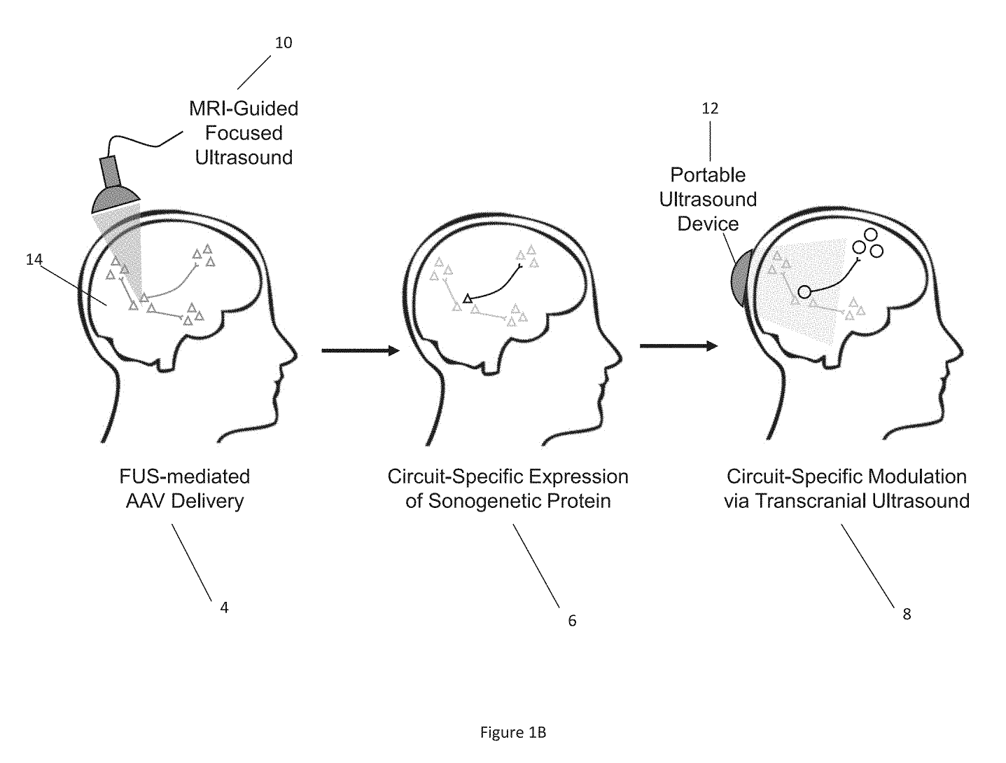

Referring to FIG. 1A, a conceptual view (2) of a configuration designed to achieve minimally-invasive, high-resolution neuromodulation of targeted tissue structures, such as deep brain structures, using circuit specific promoters combined with FUS and sonogenetics is illustrated.

FIG. 1B illustrates a process configuration (4, 6, 8) for achieving minimally-invasive, high-resolution neuromodulation. MRI-guided FUS (10) delivers the AAV to a region of the brain (14). The circuit-specific promoter identified using bioinformatics approaches expresses the sonogenetic protein in a subset of neurons i.e. the desired circuit. An external portable device (12) can deliver transcranial ultrasound to the region, which selectively activates the target circuit.

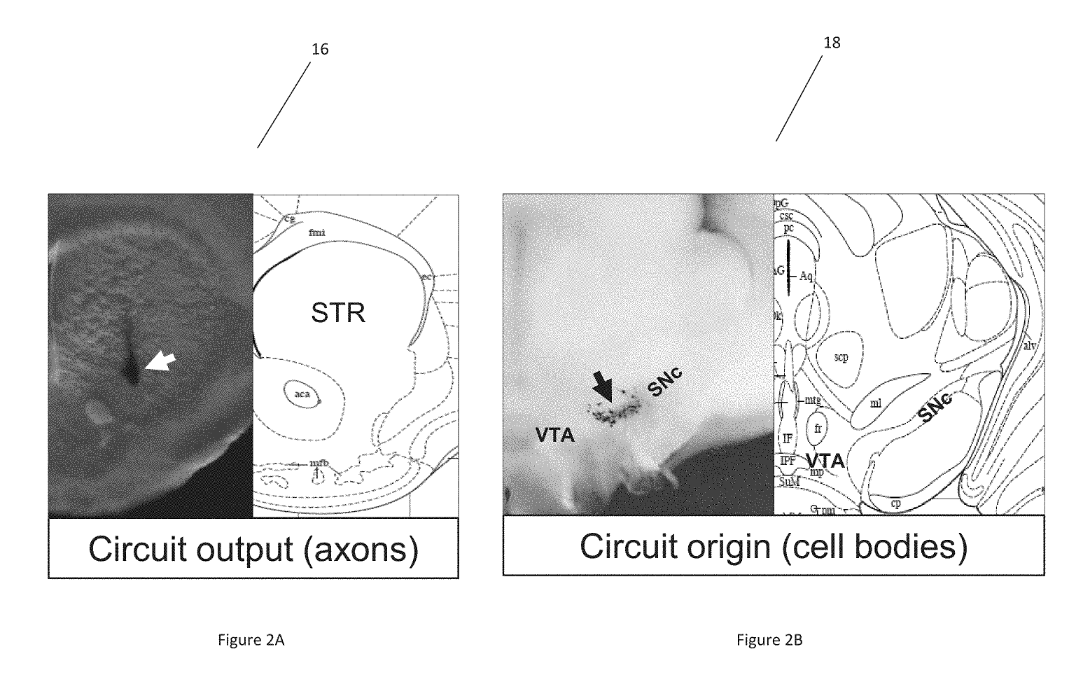

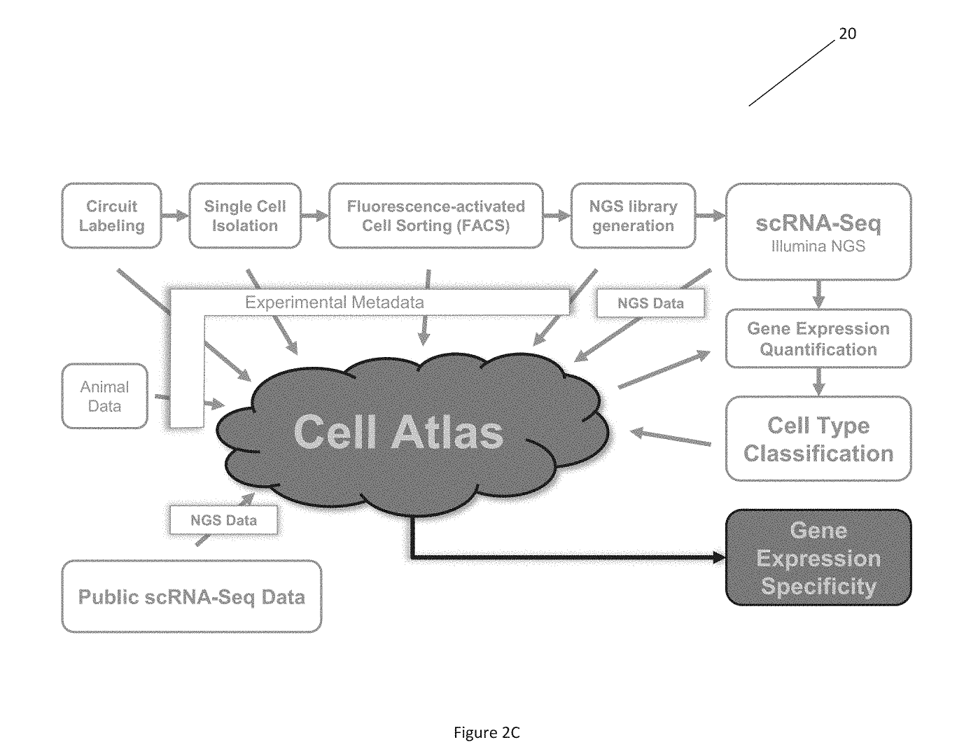

FIGS. 2A and 2B illustrate (16, 18) one embodiment of retrograde circuit labeling for analysis by single-cell genomics, wherein fluorescently labelled latex beads are injected into the circuit output at the center of the striatum (STR); then 7 days later, brains are collected and sliced by vibrating microtome. During the 7-days incubation time, latex beads are taken up in the striatum by axon terminals of dopaminergic midbrain neurons and retrogradely transported to the cell bodies (circuit origin) in the ventral midbrain, precisely, the ventral part of the substantia nigra pars compacta (SNc), close to the ventral tegmental area (VTA).

FIG. 2C illustrates an embodiment (20) for utilizing single-cell RNA sequencing data to build a cell atlas for the analysis of circuit-specific gene activity in the brain. Brain circuits may be labeled, for example as shown in FIGS. 2A and 2B, isolated and sorted. Subsequently NGS libraries may be prepared and subjected to Illumina sequencing. Using bioinformatics tools, gene expression may be quantified for each gene in the genome and in thousands to millions of cells. Cell types may be identified and classified to form the basis of a cell atlas, which integrates additional experimental metadata as well as publicly available single-cell genomics data. Finally, this cell atlas may be utilized as a platform to find genes that are expressed in highly specific neural circuits.

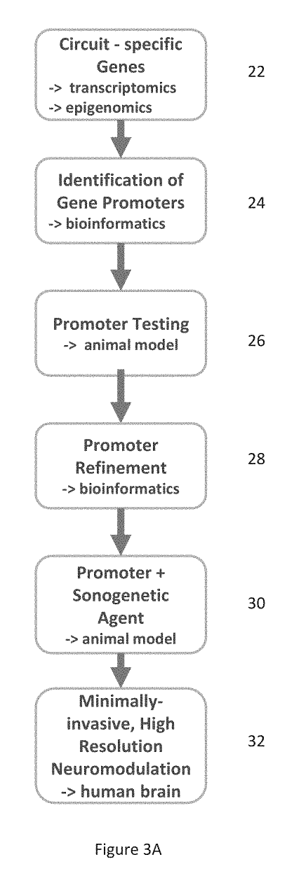

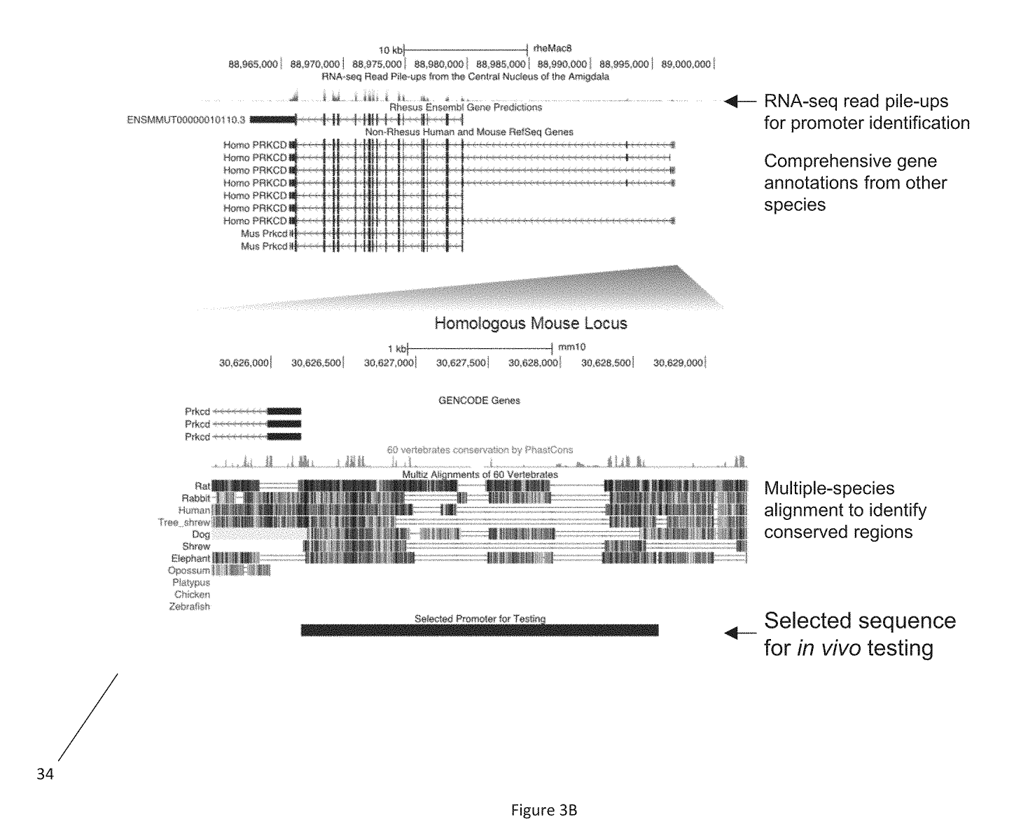

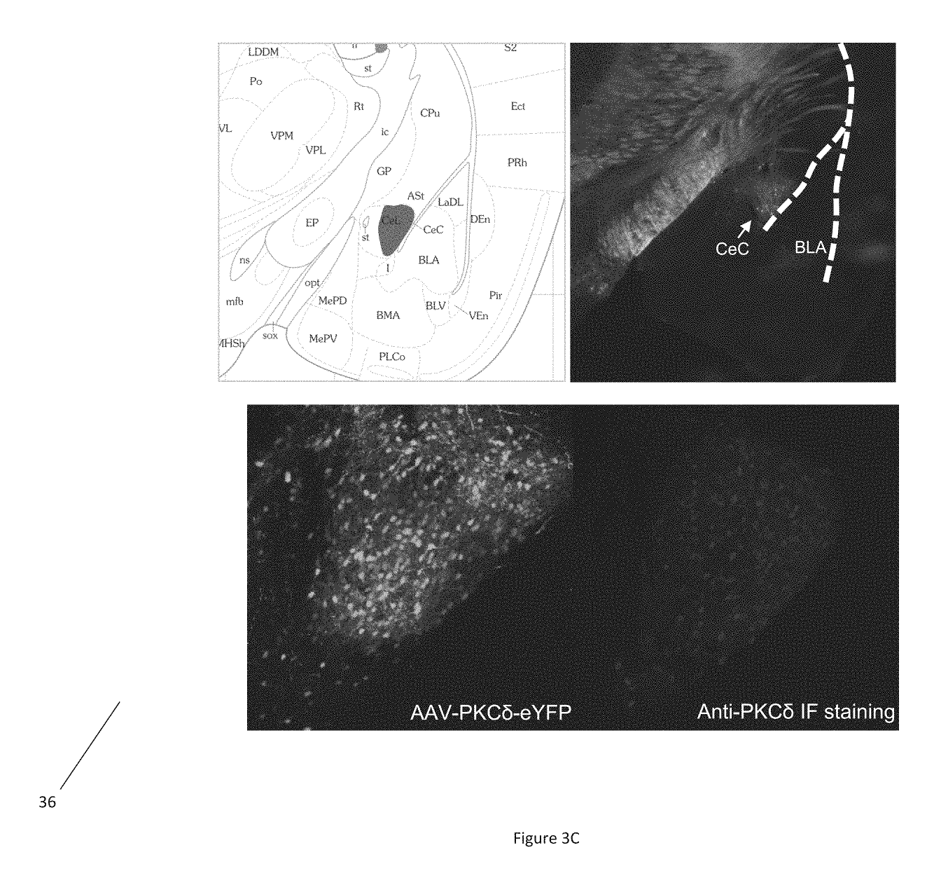

FIG. 3A illustrates one embodiment of a work flow for development of therapeutic tools, based on gene expression specificity in neural circuits of the brain: Discovery of cell type specific promoters uses information of gene expression specificity. The workflow illustrates steps for using scRNA-Seq data to determine the most likely regions of a gene comprising the promoter and critical regulatory elements that, if isolated, may be used to build a vector system for circuit-specific expression of a gene therapeutic tool (e.g. an sonogenetic protein for minimally-invasive, high-resolution neuromodulation). Referring to FIG. 3A, such steps may include, but are not limited to: circuit specific genetic analysis (22); identification of gene promoters (24—see also FIG. 3B); promoter testing (26—see also FIG. 3C); promoter refinement (28); promoter and sonogenetic agent testing (30); and minimally invasive, high resolution neuromodulation, such as in the human brain (32).

FIG. 3B illustrates (34) RNA sequencing reads which may be used to generate so-called read pile-ups across the circuit-specific gene Prkcd, specific to neurons of the central amygdala (CeC). This facilitates identification of the predominantly used transcriptional start site (TSS) for the gene in the circuit of interest. Regions upstream of the TSS then may be compared to the homologous genes from related species (e.g. Prkcd in rat and human) to determine evolutionary conserved regions that might encode critical information for the gene to be expressed specifically in the circuit of interest.

FIG. 3C illustrates (36) a scenario wherein an identified upstream region is isolated and tested (coupled to a fluorescent reporter transgene) in vivo to assess its gene-regulatory activity and specificity, in this case Prkcd promoter activity specifically in cells of the central amygdala. Top: overview of a coronal section including the central amygdala (CeC) after injection of the larger brain region with a Prkcd-promoter driven EYFP fluorescent reporter transgene. Bottom: high-resolution immune-histochemical analysis comparing endogenous Prkcd protein expression with the Prkcd-promoter driven EYFP reporter in the central amygdala.

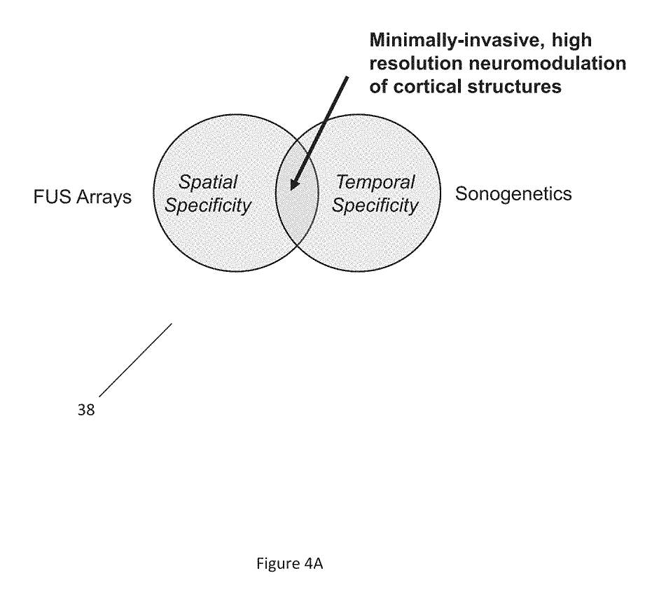

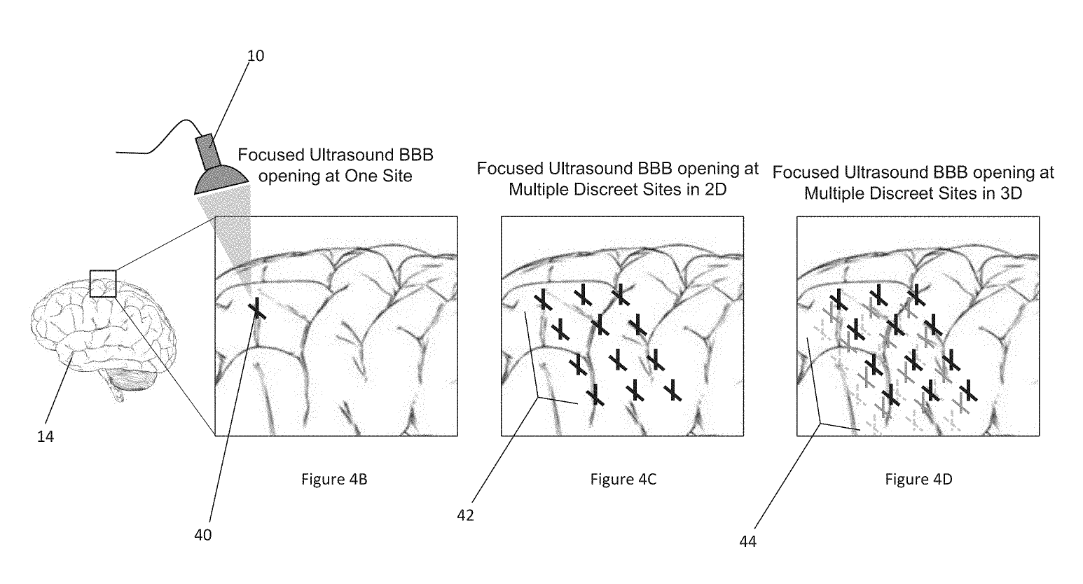

FIG. 4A illustrates a conceptual view (38) of a configuration for achieving minimally-invasive, high-resolution neuromodulation of the cortex using focused ultrasound arrays. The spatial specificity may be achieved through focused ultrasound arrays across the cortex.

FIG. 4B illustrates one embodiment of a method and configuration for utilizing focused ultrasound, such as via MRI-guidance (10), to create a BBB access point (40) at one site for one or more sonogenetic proteins. Referring to FIG. 4C, the BBB may be opened at multiple discreet sites (42) to give the desired 2-Dimensional or 3-Dimensional profile or “plan array” of sites or points, such as one which may define a surface geometry, such as a surface geometry which may be matched to the subject anatomy, for example, utilizing images associated with MRI imaging and guidance. In other words, facilitating diffusive access to the targeted neuroanatomy using focused ultrasound energy may comprise utilizing magnetic resonance data to assist in guiding an emitter of focused ultrasound energy. A plan array of focused ultrasound energy delivery points is created based at least in part upon one or more preoperative images. The plan array comprises a plurality of access points that are non-coplanar, or, for example, are in a geometric arrangement that may be selected to conform to a particular tissue contour or surface. The plan array may comprise a plurality of access points that are deemed to represent an “access surface” or “access point matrix”. Referring to FIG. 4D, an access surface may be replicated in a direction orthogonal to the access surface to create a multi-layered “access volume” (44).

An external device then may be utilized to modulate the cortex at discreet locations in high resolution (i.e., focused ultrasound energy may be applied to the patient in accordance with the plan array after completion of the plan array).

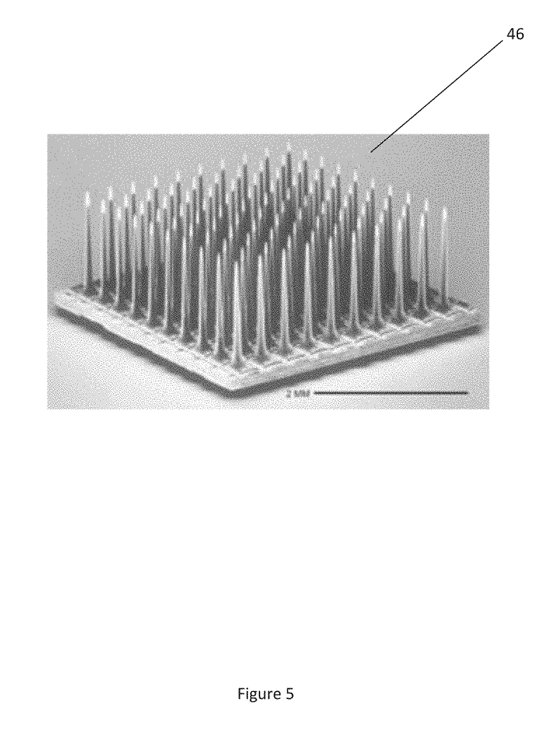

FIG. 5 illustrates an embodiment of a 96 channel electrode array, with an electrode configuration that may be replicated using focused ultrasound arrays in a manner as described above, for example, in reference to FIGS. 4C and 4D.

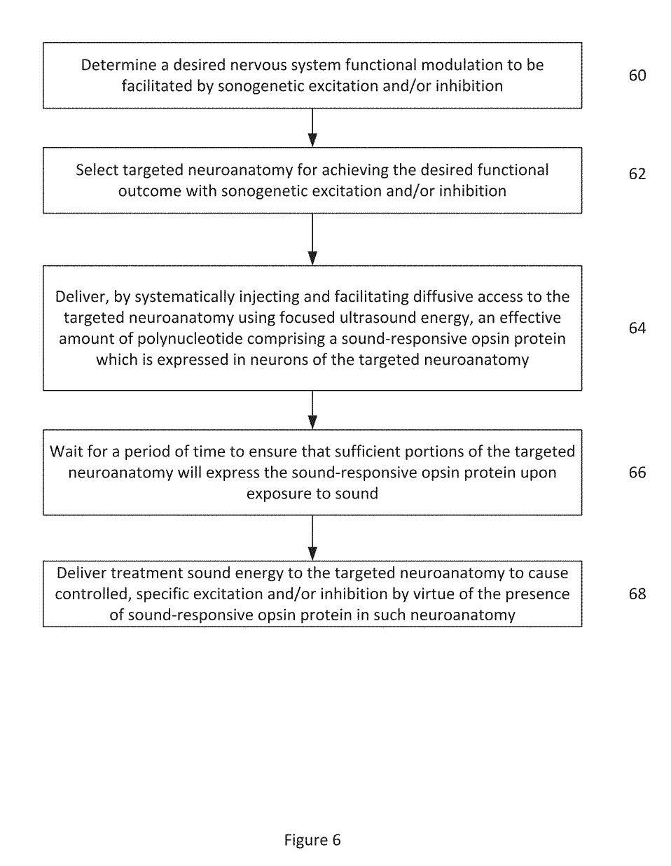

Referring to FIG. 6, in one embodiment, one may determine a desired nervous system functional modulation to be facilitated by sonogenetic excitation and/or inhibition (60); select targeted neuroanatomy for achieving the desired functional outcome with sonogenetic excitation and/or inhibition (62); deliver, such as by systematically injecting and facilitating diffusive access to the targeted neuroanatomy using focused ultrasound energy, which may be guided, at least in part, for example, utilizing MRI, an effective amount of polynucleotide comprising a sound-responsive opsin protein which is expressed in neurons of the targeted neuroanatomy (64); wait for a period of time to ensure that sufficient portions of the targeted neuroanatomy will express the sound-responsive opsin protein upon exposure to sound (66); and deliver treatment sound energy to the targeted neuroanatomy to cause controlled, specific excitation and/or inhibition by virtue of the presence of sound-responsive opsin protein in such neuroanatomy (68).

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. For example, one with skill in the art will appreciate that one or more lubricious coatings (e.g., hydrophilic polymers such as polyvinylpyrrolidone-based compositions, fluoropolymers such as tetrafluoroethylene, hydrophilic gel or silicones) may be used in connection with various portions of the devices, such as relatively large interfacial surfaces of movably coupled parts, if desired, for example, to facilitate low friction manipulation or advancement of such objects relative to other portions of the instrumentation or nearby tissue structures. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

What is claimed:

1. A method for treating the nervous system of a patient, comprising:
a. determining a desired nervous system functional modulation to be facilitated by sonogenetic intervention;
b. selecting targeted neuroanatomy for achieving the desired functional outcome with sonogenetic intervention; and
c. delivering an effective amount of polynucleotide comprising a sound-responsive opsin protein which is expressed in neurons of the targeted neuroanatomy, wherein delivering comprises systemically injecting the effective amount of polynucleotide and facilitating diffusive access to the targeted neuroanatomy using focused ultrasound energy.

2. The method of claim 1, further comprising waiting for a period of time to ensure that sufficient portions of the targeted neuroanatomy will express the sound-responsive opsin protein upon exposure to treatment sound energy.

3. The method of claim 1, further comprising delivering treatment sound energy to the targeted neuroanatomy to cause controlled, specific excitation and/or inhibition by virtue of the presence of sound-responsive opsin protein in such neuroanatomy.

4. The method of claim 1, wherein determining a desired nervous system functional modulation to be facilitated by sonogenetic intervention comprises determining a desired nervous system functional modulation to be facilitated by sonogenetic excitation.

5. The method of claim 1, wherein determining a desired nervous system functional modulation to be facilitated by sonogenetic intervention comprises determining a desired nervous system functional modulation to be facilitated by sonogenetic inhibition.

6. The method of claim 1, wherein determining a desired nervous system functional modulation to be facilitated by sonogenetic intervention comprises determining a desired nervous system functional modulation to be facilitated by sonogenetic excitation and inhibition.

7. The method of claim 1, wherein selecting targeted neuroanatomy for achieving the desired functional outcome with sonogenetic intervention comprises selecting targeted neuroanatomy for achieving the desired functional outcome with sonogenetic excitation.

8. The method of claim 1, wherein selecting targeted neuroanatomy for achieving the desired functional outcome with sonogenetic intervention comprises selecting targeted neuroanatomy for achieving the desired functional outcome with sonogenetic inhibition.

9. The method of claim 1, wherein selecting targeted neuroanatomy for achieving the desired functional outcome with sonogenetic intervention comprises selecting targeted neuroanatomy for achieving the desired functional outcome with sonogenetic excitation and inhibition.

10. The method of claim 1, wherein systemically injecting the effective amount of polynucleotide comprises injecting the effective amount of polynucleotide into a major blood vessel of the patient.

11. The method of claim 1, wherein facilitating diffusive access to the targeted neuroanatomy using focused ultrasound energy comprises utilizing magnetic resonance data to assist in guiding an emitter of focused ultrasound energy.

12. The method of claim 11, wherein a plan array of focused ultrasound energy delivery points is created based at least in part upon one or more preoperative images.

13. The method of claim 12, wherein focused ultrasound energy is applied to the patient in accordance with the plan array after completion of the plan array.

14. The method of claim 12, wherein the plan array comprises a plurality of access points that are non-coplanar.

15. The method of claim 14, wherein the plan array comprises a plurality of access points that represent an access surface.

16. The method of claim 15 wherein the access surface is replicated in a direction orthogonal to the access surface to create a multi-layered access volume.

17. The method of claim 1, wherein delivering an effective amount of polynucleotide comprises delivering a protein.

18. The method of claim 17, wherein delivering an effective amount of polynucleotide further comprises packaging the protein with a viral vector.

19. The method of claim 18, wherein the virus is selected from the group consisting of: AAV1, AAV2, AAV4, AAV5, AAV6, AAV7, AAV8, AAV9, lentivirus, and HSV.

20. The method of claim 17, wherein delivering an effective amount of polynucleotide further comprises packaging the protein with a promoter.

21. The method of claim 20, wherein the transcription promoter is selected from the group consisting of: CaMKIIa, hSyn, CMV, Hb9Hb, Thy1, and Ef1a.
